(19) United States
(12) Patent Application Publication
(10) Pub. No.: US 2016/0089326 A1
(43) Pub. Date: Mar. 31, 2016

(54) Composition for deodorizing breath and beverage made therefrom

(71) Applicant: Elias Chavando, Sherman Oaks, CA (US)

(72) Inventor: Elias Chavando, Sherman Oaks, CA (US)

(21) Appl. No.: 14/851,481

(22) Filed: Sep. 11, 2015

Related U.S. Application Data
(63) Continuation of application No. PCT/US2014/026810, filed on Mar. 14, 2013.
Application No. PCT/US2014/026810 is a continuation of application No. 13/830,340, filed on Mar. 14, 2013, now abandoned.

(51) Int. Cl. A61K 8/97; A23L 2/52; A23L 1/00; A61Q 11/00; A23L 1/30

(52) U.S. Cl. CPC A61K 8/97; A61Q 11/00; A23L 1/30; A23L 1/0017; A23L 2/52; A61K 2800/30; A61K 2800/70; A61K 2800/5922; A23V 2002/00

(57) ABSTRACT

A composition comprising an odor-suppressing mixture including parsley, alfalfa, oregano, and wheatgrass that cures halitosis. The odor-suppressing mixture comprises at least 50 weight percent of the composition. The composition is devoid of artificial preservatives, alcohol, sodium, sucrose and artificial sugar substitutes, and the odor-suppressing mixture may include the additional ingredients mint, spearmint, or thyme. A beverage is made from this composition.

### RELATED PATENT APPLICATIONS & INCORPORATION BY REFERENCE

This is a U. S. continuation patent application that claims priority of International Application No. PCT/US14/26810, filed Mar. 13, 2014, pursuant to the Patent Cooperation Treaty that in turn claimed priority based on U.S. Utility patent application Ser. No. 13/830,340, entitled “ COMPOSITION FOR DEODORIZING BREATH,” filed Mar. 14, 2013. Any and all U.S. patents, U.S. patent applications, and other documents, hard copy or electronic, cited or referred to in these applications are incorporated herein by reference and made a part of this application.

### DEFINITIONS

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

The words “consisting,” “consists of,” and other forms thereof, are intended to be equivalent in meaning and be closed ended in that an item or items following any one of these words is meant to be an exhaustive listing of such item or items and limited to only the listed item or items.

### BACKGROUND

Bad breath, commonly called halitosis, is a challenging problem. There are many breath fresheners, but many contain undesirable sugar or artificial sweeteners. Many of these breath fresheners simply mask the bad order. They do not attack the gastrointestinal source of the bad breath problem.

### SUMMARY

My breath freshening composition comprises natural occurring herbal products rich in chlorophyll. The herbal products parsley, alfalfa, oregano, and wheatgrass are mixed together to provide an odor-suppressing mixture that tastes good. My breath freshening composition is effective in humans and some domestic pets including dogs and cats; however, it may take longer to be effective depending on the person's diet, health condition, age, gender, etc.

### DETAILED DESCRIPTION OF SOME ILLUSTRATIVE EMBODIMENTS

### Odor-suppressing Mixture

The ingredients of the odor-suppressing mixture must include all four of the following herbal products, which are rich in chlorophyll:

### Leaf Parsley

The two main groups of parsley used as herbs are curly leaf (i.e.) (P. crispum crispum group; syn. P. crispum var. crispum) and Italian, or flat leaf (P. crispum neapolitanum group; syn. P. crispum var. neapolitanum); of these, the neapolitanum group more closely resembles the natural wild species.

### Alfalfa

Alfalfa is a perennial forage legume that normally lives 4-8 years, but can live more than twenty years, depending on variety and climate.

### Oregano

Oregano (US pron.: / [symbol] / or UK / [symbol] /), scientifically named Origanum vulgare by Carolus Linnaeus, is a common species of Origanum, a genus of the mint family (Lamiaceae). It is native to warm-temperate western and southwestern Eurasia and the Mediterranean region. Oregano is a perennial herb, growing from 20-80 cm tall, with opposite leaves 1-4 cm long.

### Wheatgrass

Wheatgrass is a food prepared from the cotyledons of the common wheat plant, Triticum aestivum. It is sold either as a juice or powder concentrate. Wheatgrass differs from wheat malt in that it is served freeze-dried or fresh, while wheat malt is convectively dried. Wheatgrass is also allowed to grow longer than malt is. It provides chlorophyll, amino acids, minerals, vitamins, and enzymes.

### Relative Amounts of Herbal Products

The relative amounts of these herbal products is important. Over 40 weight percent of the odor-suppressing mixture is parsley, typically from about 40 to about 55 weight percent, over 10 weight percent of the odor-suppressing mixture is alfalfa, typically from about 10 to about 15 weight percent, and less than 10 weight percent of the odor-suppressing mixture is a blend of oregano and wheatgrass, typically from about 4 to about 10 weight percent. The ratio of wheatgrass to oregano in the blend is 2 to 5 parts wheatgrass to 1 part oregano.

One embodiment of my composition in it simplest form is just the mixture itself of the herbal products in the relative amounts discussed above. A composition of these raw herbal products ground up and in the above specified amounts functions as a breath freshener when chewed by a user. Other forms include (1) a chewable tablet, (2) a chewable tablet coated with flavor, peppermint oil or other mint oil, (3) a dissolvable tablet that can be dissolved in water or any fruit juice, (4) an effervescent tablet, or (5) making a tea or other beverage from the odor-suppressing mixture.

One very convenient and appealing embodiment is a pill form. This may be a capsule or tablet, or chewable tablet comprising a binder or carrier and the odor-suppressing mixture. Suitable binders or carriers are dextrose, microcrystalline cellulose, peppermint oil, hydroxypropylcellulose, magnesium stearate and silicon dioxide. When in a pill form, the odor-suppressing mixture comprises over 50 weight percent of the ingredients in the pill. The carrier or delivery method does not affect the ability of my composition to suppress order. Its herbal products can be dry, powder, fresh or liquid. What makes the odor-suppressing mixture work is the combination of its ingredients in the approximate relative amounts discussed above.

The odor-suppressing mixture is devoid of artificial preservatives, alcohol, sodium, sucrose and artificial sugar substitutes, and it may also include naturally occurring sweeteners or other ingredients such as, for example, mint, spearmint, and thyme. These ingredients are blended together in the conventional manner to make, for example, to make the following 300 milligram tablet:

### EXAMPLE

| Ingredient | Mg | Wt. Percentage |
|---|---|---|
| Parsley | 130.25 | 43.42% |
| Alfalfa | 35.43 | 11.81% |
| Oregano | 3.5 | 1.17% |
| Mint | 45.96 | 15.32% |
| Spearmint | 75.25 | 25.08% |
| Thyme | 2.59 | 0.86% |
| Wheatgrass | 7.02 | 2.34% |
| Total | 300 | 100.00% |

### SCOPE OF THE INVENTION

The above presents a description of the best mode I contemplate of carrying out my composition and beverage and of the manner and process of making and using them, in such full, clear, concise, and exact terms as to enable a person skilled in the art to make and use. My composition and beverage are, however, susceptible to modifications and alternate constructions from the illustrative embodiments discussed above which are fully equivalent. Consequently, it is not the intention to limit my composition and beverage to the particular embodiments disclosed. On the contrary, my intention is to cover all modifications and alternate constructions coming within the spirit and scope of my composition and beverage as generally expressed by the following claims, which particularly point out and distinctly claim the subject matter of my invention:

1. A breath freshener composition devoid of artificial preservatives, alcohol, sodium, sucrose and artificial sugar substitutes, and said composition including an odor-suppressing mixture including parsley, alfalfa, oregano, and wheatgrass that comprises at least 50 weight percent of the composition.

2. The breath freshener composition of claim 1 where odor-suppressing mixture includes one or more ingredients selected from the group consisting of mint, spearmint, and thyme.

3. The breath freshener composition of claim 1 comprising
from about 40 to about 55 weight percent parsley,
from about 10 to about 15 weight percent alfalfa, and
from about 4 to about 10 weight percent a blend of oregano and wheatgrass,
the ratio of wheatgrass to oregano in the blend being 2 to 5 parts wheatgrass to 1 part oregano.

4. A breath freshener composition devoid of artificial preservatives, alcohol, sodium, sucrose and artificial sugar substitutes, said composition comprising
at least 50 weight percent of the composition an odor-suppressing mixture including from about 40 to about 55 weight percent parsley, from about 10 to about 15 weight percent alfalfa, and from about 4 to about 10 weight percent of a blend of oregano and wheatgrass,
the ratio of wheatgrass to oregano in the blend being 2 to 5 parts wheatgrass to 1 part oregano.

5. The breath freshener composition of claim 4 where odor-suppressing mixture includes one or more ingredients selected from the group consisting of mint, spearmint, and thyme.

6. A breath freshener composition comprising an odor-suppressing mixture of natural occurring herbal products containing chlorophyll including parsley, alfalfa, oregano, and wheatgrass, said odor-suppressing mixture comprising at least 50 weight percent of the composition and being devoid of artificial preservatives, alcohol, sodium, sucrose and artificial sugar substitutes.

7. The breath freshener composition of claim 6 where odor-suppressing mixture includes one or more ingredients selected from the group consisting of mint, spearmint, and thyme.

8. A tablet devoid of artificial preservatives, alcohol, sodium, sucrose and artificial sugar substitutes and comprising
approximately 43 weight percent parsley,
approximately 11 weight percent alfalfa,
approximately 1 weight percent oregano,
approximately 2 weight percent wheatgrass,
approximately 15 weight percent mint,
approximately 25 weight percent spearmint, and
approximately 1 weight percent thyme.

9. The tablet of claim 8 in the form of (1) a chewable tablet, (2) a chewable tablet with flavor coating, (3) a dissolvable tablet that can be dissolved in water or any fruit juice, or (4) an effervescent tablet.

10. A beverage made from an odor-suppressing mixture including parsley, alfalfa, oregano, and wheatgrass.
